(19) United States
(12) Patent Application Publication
(10) Pub. No.: US 2024/0082160 A1
(43) Pub. Date: Mar. 14, 2024

(54) AMLODIPINE DRY SUSPENSION AND PREPARATION METHOD THEREFOR

(71) Applicants: GUANGZHOU APICHOPE PHARMACEUTICAL CO., LTD., Guangzhou, Guangdong (CN); GUANGDONG ZERUI PHARMACEUTICAL CO., LTD., Guangzhou, Guangdong (CN); GUANGZHOU UNIRISE PHARMACEUTICAL CO., LTD., Guangzhou, Guangdong (CN); GUANGZHOU RUNLIN PHARMACEUTICAL TECHNOLOGY CO., LTD., Guangzhou, Guangdong (CN); APICHOPE BIO PHARMACEUTICAL CO., LTD., Guangzhou, Guangdong (CN)

(72) Inventors: Hanxiong Li, Guangzhou, Guangdong (CN); Xianzhu Wang, Guangzhou, Guangdong (CN); Weitang Chen, Guangzhou, Guangdong (CN); Yuan Chen, Guangzhou, Guangdong (CN); Ping Guo, Guangzhou, Guangdong (CN); Yongyi Wu, Guangzhou, Guangdong (CN); Yongmei Qin, Guangzhou, Guangdong (CN); Hongxue Huang, Guangzhou, Guangdong (CN); Li Wang, Guangzhou, Guangdong (CN)

(21) Appl. No.: 18/272,796

(22) PCT Filed: Dec. 31, 2021
(86) PCT No.: PCT/CN2021/143500

§ 371 Date: Jul. 18, 2023

(30) Foreign Application Priority Data
Jan. 18, 2021 (CN) 202110063061.2

(51) Int. Cl. A61K 9/16; A61K 31/4418

(52) U.S. Cl. CPC A61K 9/1694; A61K 9/1617; A61K 31/4418

(57) ABSTRACT

An amlodipine dry suspension, comprising a diluent, a suspending aid, etc. and being free of surfactants. A preparation method therefor comprises the following steps: a) preparing an aqueous mixture 1 containing amlodipine besylate by using a high-speed homogenization method; b) adding sodium benzoate to the mixture 1, and homogenizing same at a high speed to obtain a mixture 2; and c) granulating the mixture 2 and other components, and then drying same. The raw materials are homogenized and mixed at a high speed, no surfactant is added during the preparation, amlodipine is fully wetted by using a high-speed homogenization process, prescription components are simplified, and the amlodipine dry suspension is more suitable for children to use. Compared with ultrasonic stirring, scaled-up and industrial production are easier to realize for the high-speed homogenization process, and at the same time, the preparation is good in stability and convenient to carry.

This application claims the priority of CN Application No. 202110063061.2, filed Jan. 18, 2021, which is hereby incorporated by reference in its entirety.

### FIELD OF THE INVENTION

The invention belongs to the technical field of amlodipine preparation, and more particularly relates to a dry suspension of amlodipine and a preparation method thereof.

### BACKGROUND OF THE INVENTION

Amlodipine is a calcium channel blocker, which is used in the treatment of hypertension, coronary heart disease, etc. amlodipine has been widely used clinically for many years, with definite efficacy, and is one of the most frequently used antihypertensive drugs.

The most widely used dosage form of amlodipine is oral tablet, such as Norvasc. However, up to a quarter of the population has difficulty ingesting and swallowing solid dosage forms, and this segment of the population is poorly compliant with solid dosage forms of drug, making therapy ineffective. In addition, solid dosage forms are generally unfriendly to children or the elderly due to the increased risk of suffocation; additionally, the dose of amlodipine to a child is calculated based on the child's weight, when the calculated dose differs from one or more intact solids, the solid dosage form must be separated to provide the correct dose, when solid tablets are applied to children, it is difficult to ensure that the dosage is administered accurately.

Patents WO2018067959A1 and WO2019200143A1 disclose an oral liquid formulation of amlodipine, and the corresponding patented product amlodipine suspension (KATERZIA®, AZURITY PHARMACEUTICALS INC) has been listed in the United States, which has solved the technical problems of amlodipine solid preparation for some patients, such as difficulty in ingestion and swallowing, and difficulty in dose splitting, providing patients with higher dose accuracy, compliance, and availability of preparations, which can meet the needs of various patients (especially children and the elderly). However, the commercially available patented product amlodipine suspension (KATERZIA®, AZURITY PHARMACEUTICALS INC) has poor stability, the stable storage condition is 5±5° C.. It has high requirements for storage temperature, and oral liquid formulations have disadvantages such as large volume and inconvenient carrying.

### SUMMARY OF THE INVENTION

The technical problem to be solved by the invention is to provide an oral formulation of amlodipine with no difficulty in ingestion, swallowing and dose splitting, good compliance and taste, and at the same time, convenient carrying and good stability.

In addition, the oral formulation of amlodipine provided by the invention comprises an amlodipine salt with lower solubility prepared from commercially available amlodipine besylate, which solves the problem that amlodipine besylate is not suitable for preparing dry suspension cause by slightly soluble in water.

Disclosed herein is a process for preparing amlodipine dry suspension, the process comprising: a) preparing an aqueous mixture 1 containing amlodipine besylate by using a high-speed homogenization method; b) adding sodium benzoate to the mixture 1, and homogenizing same at a high speed to obtain a mixture 2; and c) granulating the mixture 2 and other components, and then drying same.

No surfactant is added during the process. In the preparation method mentioned in the present invention, other components in step c) include: pharmaceutically acceptable diluents, suspending agents, defoamers, binders, glidants, flavoring agents, fillers, lubricating agents and other ingredients, granulated with amlodipine.

In the present invention, the amlodipine oral liquid in the prior art is changed to a dry suspension, which is easy to divide dosage and convenient to carry. Since amlodipine besylate is slightly soluble in water, it is not suitable for preparation into a dry suspension, and amlodipine benzoate suitable for preparation into a dry suspension is not available for sale as commercially available raw materials. The amlodipine dry suspension disclosed in the invention is prepared by preparing the commercially available amlodipine besylate into a salt form with lower solubility in aqueous media than amlodipine benzoate which is more suitable for preparing dry suspension formulations without changing the active ingredients of amlodipine.

Prior art WO2019200143A1 discloses: A process for preparing amlodipine benzoate, the process comprising: (i) providing an aqueous mixture comprising an amlodipine salt that is more soluble in aqueous media than amlodipine benzoate; (ii) adding sodium benzoate to the aqueous mixture to form a first mixture; and (iii) subjecting the first mixture to ultrasonic agitation thereby forming a second mixture comprising amlodipine benzoate. However, in order to achieve better technical effects and adapt to industrial production, it is necessary to add surfactants such as polysorbate 80 in each step: For example, polysorbate 80 is added to the aqueous mixture in step (i) and mixed before adding amlodipine salt (such as amlodipine besylate) that is more soluble in aqueous media than amlodipine benzoate, minimizing the adhesion of amlodipine to metal containers commonly used in production equipment, such as those made of stainless steel. In the absence of polysorbate 80, prepare the benzoate of amlodipine in a stainless steel container, amlodipine besylate will be adsorbed/adhered to the stainless steel surface, add purified water to the stainless steel container, start mixing with an agitator containing a stainless steel stirring blade, add amlodipine besylate to prepare the batch without polysorbate 80, mix the suspension for about 5 minutes, and then add sodium benzoate, Large crystals rapidly forming in the suspension will be observed, and solid materials can be observed adhering and coating on the shaft and impeller of the stainless steel stirring paddle, as well as on the inner surface of the stainless steel container. Or mixing an aqueous mixture containing amlodipine besylate and polysorbate 80 before adding sodium benzoate in step (ii); Or mixing the first mixture containing amlodipine besylate, polysorbate 80 and sodium benzoate, and then ultrasonic stirring; To ensure uniform dispersion of amlodipine besylate, which is conducive to the formation of amlodipine benzoate. When preparing amlodipine benzoate suspension, it also includes adding the second part of polysorbate 80 to the second mixture containing amlodipine benzoate after step (iii). The method further adds the total amount of water, thus facilitating the formation of amlodipine benzoate suspension.

The process for preparing amlodipine dry suspension disclosed by the invention does not add surfactant such as polysorbate 80, and the amlodipine is fully wetted by using a high-speed homogenization process, which solves the problem that the surfactant such as polysorbate 80 needs to be added in the prior art to avoid the adhesion of amlodipine and the difficulty of dispersion of amlodipine besylate, which is not conducive to the formation of amlodipine benzoate. prescription components are simplified, and the amlodipine dry suspension is more suitable for children to use. Compared with ultrasonic stirring, scaled-up and industrial production are easier to realize for the high-speed homogenization process, Moreover, the technical personnel of the present invention unexpectedly found that the stability of the finished product of the preparation was significantly improved by not adding common surfactants such as polysorbate 80, sodium lauryl sulfate.

The particle size of the API is closely related to the process for preparing dry suspension. Too large particle size or too wide particle size distribution will lead to poor sedimentation volume ratio and dose uniformity of the suspension, which are not conducive to the quality of the dry suspension. It has been verified by experiments, the high-speed homogenization process can not only fully wet amlodipine, but also solve the technical problem of stability reduction caused by the incompatibility of surfactants such as polysorbate 80 with amlodipine, and the prepared amlodipine particle size smaller and narrower in particle size distribution, the prepared dry suspension has the best effect and is suitable for large-scale industrial production; the sodium benzoate added is used for salifying amlodipine, and has the effect of preservatives, and diluents, suspending agent are added to help suspending and dispersing.

Preferably, the weight percentage of the amlodipine besylate in the dry suspension is between 0.5% to 2%, and the weight percentage of the amlodipine besylate in the aqueous mixture 1 in the step a) is between 1% and 5%; The ratio of the weight of sodium benzoate in preparation step b) to amlodipine besylate in step a) is between 1:1 and 5:1; The diluent is preferably one or a mixture of mannitol, erythritol and maltitol, and its weight percentage in the dry suspension is 80%-95%; the suspending agent is preferably hydroxypropyl methylcellulose K4M [hydroxypropyl methylcellulose is divided into four substitution types based on the different content of methoxy and hydroxypropyl groups. This product is 2208 type, K series, viscosity (2%) 2700-5040 mPa·s, molecular weight 400000, (Ch.P 2020 Edition, Part 4, the same below)], hydroxypropyl methylcellulose K750 [2208 type, K series, viscosity (2%) 560-1050 mPa·s, molecular weight 250000] One or a mixture of hydroxypropyl methylcellulose K1500 [2208 type, K series, viscosity (2%) 1125-2100 mPa·s, molecular weight 300000], with a weight percentage between 1% and 5% in the dry suspension.

In the present invention, a defoamer, such as simethicone, can also be added for defoaming to avoid excessive bubbles in the redissolution process before taking; and adding a glidant, such as colloidal silicon dioxide, silicon dioxide or talcum powder, to adjust fluidity, while avoiding agglomeration of particles; and adding flavoring agents, such as sucralose, sodium saccharin or aspartame, to adjust the taste. Binders such as hypromellose E5 [Type 2910, E series, viscosity (2%) 4-6 mPa·s] are convenient for granulation.

The process step c) is preferably high shear wet granulation or fluidized bed granulation.

Most preferably, the diluent is mannitol, the suspension agent is hypromellose K4M, and the binder is hypromellose E5. Among them, mannitol has good taste, low reducing sugar content, good compatibility with amlodipine, and does not affect the stability of the product. Hypromellose K4M has good suspension effect and easy to disperse, which is conducive to the redissolution of the product before taking.

The present invention provides a kind of preferred formulation as follows:

| Component | amount per bottle (g) |
|---|---|
| Amlodipine besylate | 0.1~0.5 |
| Sodium benzoate | 0.1~0.5 |
| Purified water ^{a} | 2.5~5 |
| Mannitol | 10~20 |
| Hypromellose K4M | 0.3~0.5 |
| Hypromellose E5 | 0.05~0.15 |
| Simethicone | 0.01~0.02 |
| Purified water ^{b} | 2~5 |
| Sucralose | 0.05~0.15 |
| Colloidal silicon dioxide | 0.03~0.07 |

The present invention also provides a specific formulation and preparation method therefor as follows:

| Component | amount per bottle (g) |
|---|---|
| Amlodipine besylate | 0.139 |
| Sodium benzoate | 0.5 |
| Purified water ^{a} | 3.34 |
| Mannitol | 13.721 |
| Hypromellose K4M | 0.375 |
| Hypromellose E5 | 0.1 |
| Simethicone | 0.015 |
| Purified water ^{b} | 2 |
| Sucralose | 0.1 |
| Colloidal silicon dioxide | 0.05 |

^{a} Adding amlodipine besylate to purified water ^{a}, preparing by using a high-speed homogenization method, to make the dispersion uniform, obtain a mixture 1;
^{b} Adding sodium benzoate to the mixture 1, and homogenizing same at a high speed to obtain a mixture 2;
^{c} Putting mannitol, hypromellose K4M and simethicone into the wet mixing granulator, and adding hypromellose E5 aqueous solution, granulating, and drying and sieving to obtain blank granules, and then spraying the mixture 2 prepared by step b) for one-step granulation, Sieving, and adding sucralose and colloidal silicon dioxide, then final blending and packaging.

After dispersion with water, the particle size range is preferably as follows: D90≤70 um, 10 um≤D50≤50 um.

The amlodipine dry suspension disclosed in the present invention has simple and safe ingredients, at the same time, the preparation is good in stability, easy to product and convenient to carry. So the preparation has good market prospects.

### DETAILED DESCRIPTION OF THE PREFERRED EMBODIMENTS

The above-mentioned content of the present invention will be further described in detail below through specific embodiments. However, it should not be understood that the scope of the above-mentioned subject of the present invention is limited only to the following embodiments. Without departing from the above technical idea of the present invention, various substitutions or changes made based on common technical knowledge in the field and conventional means should all be included in the scope of the present invention.

### Example 1

### Formulation:

| Component | amount |
|---|---|
| Amlodipine besylate | 13.9 g |
| Sodium benzoate | 13.9 g |
| Purified water ^{a} | 1155.0 g |
| Mannitol | 621.1 g |
| Hypromellose K4M | 13.0 g |
| Purified water ^{b} | 200.0 g |

The preparation process is as follows:
^{a} Adding the prescribed amount of amlodipine besylate to purified water ^{a}, preparing by using a high-speed homogenization method, the rotating speed is 5000 RPM, and the homogenization is performed for 2 minutes to make the dispersion uniform, obtain a mixture 1;
^{b} Adding sodium benzoate to the mixture 1, preparing by using a high-speed homogenization process, the rotating speed is 10000 RPM, and the homogenization is performed for 10 minutes, to obtain a mixture 2 for use;
^{c} Putting mannitol and hypromellose K4M into the wet mixing granulator, and adding purified waterb, granulating, and drying and sieving to obtain blank granules; then adding the blank granules to the fluidized bed granulating dryer, and spraying the mixture 2 prepared by step b) for one-step granulation;
^{d} Sieving and final blending;
^{e} Packaging: each bottle contains amlodipine 100 mg.

### Example 2

### Formulation:

| Component | amount |
|---|---|
| Amlodipine besylate | 13.9 g |
| Sodium benzoate | 28 g |
| Purified water ^{a} | 250.2 g |
| Ethanol | 27.8 g |
| Erythritol | 677.1 g |
| Hypromellose K1500 | 31.9 g |
| Simethicone | 1.0 g |

The preparation process is as follows:
^{a} Adding the prescribed amount of amlodipine besylate to the mixed solvent of purified water ^{a} and ethanol, preparing by using a high-speed homogenization method, the rotating speed is 8000 RPM, and the homogenization is performed for 2 minutes to make the dispersion uniform, obtain a mixture 1;
^{b} Adding sodium benzoate to the mixture 1, preparing by using a high-speed homogenization process, the rotating speed is 8000 RPM, and the homogenization is performed for 10 minutes, to obtain a mixture 2 for use;
^{c} Putting erythritol, hypromellose K1500 and simethicone into the fluidized bed granulating dryer and spraying the mixture 2 prepared by step b) for one-step granulation;
^{d} Sieving and final blending;
^{e} Packaging: each bottle contains amlodipine 100 mg.

### Example 3

### Formulation:

| Component | amount |
|---|---|
| Amlodipine besylate | 13.9 g |
| Sodium benzoate | 66.8 g |
| Purified water ^{a} | 334 g |
| Maltitol | 1488.0 g |
| Erythritol | 600.1 |
| Hypromellose K750 | 33.2 g |
| Simethicone | 1.3 g |
| Aspartame | 15 g |
| talcum powder | 4.2 g |

The preparation process is as follows:
^{a} Adding the prescribed amount of amlodipine besylate to purified water ^{a}, preparing by using a high-speed homogenization method, the rotating speed is 8000 RPM, and the homogenization is performed for 2 minutes to make the dispersion uniform, obtain a mixture 1;
^{b} Adding sodium benzoate to the mixture 1, preparing by using a high-speed homogenization process, the rotating speed is 8000 RPM, and the homogenization is performed for 10 minutes, to obtain a mixture 2 for use;
^{c} Putting maltitol, erythritol, hypromellose K750, and simethicone into the fluidized bed granulating dryer, and spraying the mixture 2 prepared by step b) for one-step granulation;
^{d} Sieving, and adding aspartame and talcum powder, then final blending;
^{e} Packaging: each bottle contains amlodipine 100 mg.

### Example 4

### Formulation:

| Component | amount |
|---|---|
| Amlodipine besylate | 13.9 g |
| Sodium benzoate | 31.9 g |
| Purified water ^{a} | 310 g |
| Maltitol | 501.1 g |
| Mannitol | 211.7 |
| Hypromellose K4M | 14.2 g |
| Hypromellose K750 | 15.1 g |
| Hypromellose EXF | 10 g g |
| Simethicone | 1.3 g |
| Purified water ^{b} | 200 g |
| Colloidal silicon dioxide | 3.6 g |

The preparation process is as follows:
^{a} Adding the prescribed amount of amlodipine besylate to purified water ^{a}, preparing by using a high-speed homogenization method, the rotating speed is 8000 RPM, and the homogenization is performed for 2 minutes to make the dispersion uniform, obtain a mixture 1;
^{b} Adding sodium benzoate to the mixture 1, preparing by using a high-speed homogenization process, the rotating speed is 8000 RPM, and the homogenization is performed for 10 minutes, to obtain a mixture 2 for use;
^{c} Putting maltitol, mannitol, Hypromellose K4M, hypromellose K750 and simethicone into a fluidized bed granulating dryer, and adding hypromellose EXF to purified water ^{b} to dissolve as a binder, After being dissolved, spraying into the fluidized bed granulating dryer, and then spraying the mixture 2 prepared by step b ) for one-step granulation;
^{d} Sieving, and adding colloidal silicon dioxide, then final blending;
^{e} Packaging: each bottle contains amlodipine 100 mg.

### Example 5

### Formulation:

| Component | amount |
|---|---|
| Amlodipine besylate | 13.9 g |
| Sodium benzoate | 66.8 g |
| Purified water ^{a} | 334 g |
| Mannitol | 1377.8 g |
| Hypromellose K750 | 36.8 g |
| Hypromellose E5 | 10 g |
| Simethicone | 1.5 g |
| Purified water ^{b} | 200 g |
| Aspartame | 15 g |
| Talcum powder | 4.2 g |

The preparation process is as follows:
^{a} Adding the prescribed amount of amlodipine besylate to purified water ^{a}, preparing by using a high-speed homogenization method, the rotating speed is 8000 RPM, the homogenization is performed for 2 minutes to make the dispersion uniform, obtain a mixture 1;
^{b} Adding sodium benzoate to the mixture 1, preparing by using a high-speed homogenization process, the rotating speed is 15000 RPM, and the homogenization is performed for 10 minutes, to obtain a mixture 2 for use;
^{c} Putting mannitol, hypromellose K750 and simethicone into the wet mixing granulator, adding hypromellose E5 to purified water ^{b} to dissolve as a binder, granulating, and drying and sieving to obtain blank granules; then adding the blank granules to the fluidized bed granulating dryer, and spraying the mixture 2 prepared by step b) for one-step granulation;
^{d} Sieving, and adding aspartame and talcum powder, then final blending;
^{e} Packaging: each bottle contains amlodipine 100 mg.

### Example 6

### Formulation:

| Component | amount |
|---|---|
| Amlodipine besylate | 13.9 g |
| Sodium benzoate | 50 g |
| Purified water ^{a} | 413 g |
| Mannitol | 1371.6 g |
| Hypromellose K4M | 37.5 g |
| Hypromellose E5 | 10 g |
| Simethicone | 2.0 g |
| Purified water ^{b} | 200 g |
| Saccharin Sodium | 10 g |
| silicon dioxide | 4 g |

The preparation process is as follows:
^{a} Adding the prescribed amount of amlodipine besylate to purified water ^{a}, preparing by using a high-speed homogenization method, the rotating speed is 5000 RPM, the homogenization is performed for 2 minutes to make the dispersion uniform, obtain a mixture 1;
^{b} Adding sodium benzoate to the mixture 1, preparing by using a high-speed homogenization process, the rotating speed is 5000 RPM, the homogenization is performed for 20 minutes, to obtain a mixture 2 for use;
^{c} Putting mannitol and hypromellose K4M into the wet mixing granulator, adding hypromellose E5 to purified water ^{b} to dissolve as a binder, granulating, and drying and sieving to obtain blank granules; then adding the blank granules to the fluidized bed granulating dryer, and spraying the mixture 2 prepared by step b) for one-step granulation;
^{d} Sieving, and adding saccharin sodium and silicon dioxide, then final blending;
^{e} Packaging: each bottle contains amlodipine 100 mg.

### Example 7

### Formulation:

| Component | amount |
|---|---|
| Amlodipine besylate | 13.9 g |
| Sodium benzoate | 50 g |
| Purified water ^{a} | 334 g |
| Mannitol | 1372.1 g |
| Hypromellose K4M | 37.5 g |
| Hypromellose E5 | 10 g |
| Simethicone | 1.5 g |
| Purified water ^{b} | 200 g |
| Sucralose | 10 g |
| Colloidal silicon dioxide | 5 g |

The preparation process is as follows:
^{a} Adding the prescribed amount of amlodipine besylate to purified water ^{a}, preparing by using a high-speed homogenization method, the rotating speed is 5000 RPM, the homogenization is performed for 2 minutes to make the dispersion uniform, obtain a mixture 1;
^{b} Adding sodium benzoate to the mixture 1, preparing by using a high-speed homogenization process, the rotating speed is 15000 RPM, the homogenization is performed for 15 minutes, to obtain mixture a 2 for use;
^{c} Putting mannitol, Hypromellose K4M and simethicone into the wet mixing granulator, adding hypromellose E5 to purified water ^{b} to dissolve as a binder, granulating, and drying and sieving to obtain blank granules; then adding the blank granules to the fluidized bed granulating dryer, and spraying the mixture 2 prepared by step b) for one-step granulation;
^{d} Sieving, and adding sucralose and colloidal silicon dioxide, then final blending;
^{e} Packaging: each bottle contains amlodipine 100 mg.

Control group 1: Amlodipine suspension (KATERZIA®, AZURITY PHARMACEUTICALS INC)

### Control Group 2

### Formulation:

| Component | amount |
|---|---|
| Amlodipine besylate | 13.9 g |
| Sodium benzoate | 50 g |
| Polysorbate 80 | 6.7 g |
| Purified water ^{a} | 334 g |
| Mannitol | 1372.1 g |
| Hypromellose K4M | 37.5 g |
| Hypromellose E5 | 10 g |
| Simethicone | 1.5 g |
| Purified water ^{b} | 200 g |
| Sucralose | 10 g |
| Colloidal silicon dioxide | 5 g |

The preparation process is same as Example 7.

### Control Group 3

### Formulation:

| Component | amount |
|---|---|
| Amlodipine besylate | 13.9 g |
| Sodium benzoate | 50 g |
| Polysorbate 20 | 5 g |
| Purified water ^{a} | 334 g |
| Mannitol | 1372.1 g |
| Hypromellose K4M | 37.5 g |
| Hypromellose E5 | 10 g |
| Simethicone | 1.5 g |
| Purified water ^{b} | 200 g |
| Sucralose | 10 g |

The preparation process is same as Example 7.

### Control Group 4

### Formulation:

| Component | amount |
|---|---|
| Amlodipine besylate | 13.9 g |
| Sodium benzoate | 50 g |
| Sodium lauryl sulfate | 5 g |
| Purified water ^{a} | 334 g |
| Mannitol | 1372.1 g |
| Hypromellose K4M | 37.5 g |
| Hypromellose E5 | 10 g |
| Simethicone | 1.5 g |
| Purified water ^{b} | 200 g |
| Sucralose | 10 g |
| Colloidal silicon dioxide | 5 g |

The preparation process is same as Example 7.

### Control Group 5

Formulation is same as Example 7.
The preparation process is as follows:
a) Adding the prescribed amount of amlodipine besylate to purified water ^{a}, stirring until the dispersion uniform, to obtain a mixture 1;
b) Adding sodium benzoate to the mixture 1, stirring 15 minutes, to obtain a mixture 2 for use;
c) Putting mannitol, hypromellose K4M and simethicone into the wet mixing granulator, adding hypromellose E5 to purified water^{b} to dissolve as a binder, granulating, and drying and sieving to obtain blank granules;
d) Adding the blank granules to the fluidized bed granulating dryer, and spraying the mixture 2 prepared by step b) for one-step granulation;
e) Sieving, and adding sucralose and colloidal silicon dioxide, final blending and packaging.

### Control Group 6

Formulation is same as Example 7.
The preparation process is as follows:
a) Adding the prescribed amount of amlodipine besylate to purified water ^{a}, subjecting to ultrasonic agitation until the dispersion uniform, to obtain a mixture 1;
b) Adding sodium benzoate to the mixture 1, subjecting to ultrasonic agitation 15 minutes, to obtain a mixture 2 for use;
c) Putting mannitol, hypromellose K4M and simethicone into the wet mixing granulator, adding hypromellose E5 to purified water^{b} to dissolve as a binder, granulating, and drying and sieving to obtain blank granules;
d) Adding the blank granules to the fluidized bed granulating dryer, and spraying the mixture 2 prepared by step b) for one-step granulation;
e) Sieving, and adding sucralose and colloidal silicon dioxide, final blending and packaging.

The test results of the samples prepared in the above embodiments and control groups are as follows:

1, Stability test: The stability is evaluated by related substances, and the related substances are detected by HPLC. The chromatographic system is as follows: Column: Use octadecyl silane bonded silica as filler (Phenomenex Luna C18 4.6 mm×250 mm, 5 μm or equivalent column); Mobile phase A: 1% triethylamine solution (adjust pH to 2.8 with phosphoric acid), Mobile phase B: methanol-acetonitrile (70:30), performing gradient elution according to the following table; Flow rate: 1.0 mL/min; Column temperature: 30° C.: Injector temperature: 10° C. Detector: UV 237 nm; Injection size: 15 μl.

TABLE 1

| | mobile phase A | mobile phase B |
|---|---|---|
| time (min) | (%) | (%) |
| 0 | 80 | 20 |
| 40 | 20 | 80 |
| 50 | 20 | 80 |
| 50.1 | 80 | 20 |
| 65 | 80 | 20 |

The results showed that comprising of surfactants such as polysorbate 80 seriously affects the stability of the formulation, and the stability of the samples in the examples was better than that of the control groups containing wetting agents. The specific results are shown in Table 2 and Table 3.

TABLE 2
The results of the HPLC analysis for related substances in the samples stored at 60° C. for 10 days

| | | | RRT | | | | | | | | | | | | | |
|---|---|---|---|---|---|---|---|---|---|---|---|---|---|---|---|---|
| related substances | | | | | | | 0.83 | | | | | | | | | Total |
| (%) | | | 0.34 | 0.7 | 0.78 | 0.81 | (Impurity A) | 0.97 | 1.07 | 1.12 | 1.24 | 1.26 | 1.35 | 1.41 | 1.42 | impurities |
| Example 1 | 0 | d | — | — | — | — | 0.03 | — | — | — | 0.02 | — | — | — | — | 0.05 |
| | 10 | d | — | — | — | — | 0.18 | — | — | — | 0.08 | — | 0.05 | — | — | 0.31 |
| Example 2 | 0 | d | — | — | — | — | 0.05 | — | — | — | 0.02 | — | — | — | — | 0.07 |
| | 10 | d | — | — | — | — | 0.23 | — | — | — | 0.1 | — | 0.04 | — | 0.05 | 0.42 |
| Example 3 | 0 | d | — | — | — | — | 0.03 | — | — | — | 0.01 | — | 0.01 | — | — | 0.05 |
| | 10 | d | — | — | — | — | 0.2 | 0.02 | — | — | 0.09 | — | 0.06 | — | — | 0.37 |
| Example 4 | 0 | d | — | — | — | — | 0.1 | — | — | — | 0.1 | — | 0.01 | — | — | 21 |
| | 10 | d | — | — | — | — | 0.32 | 0.05 | — | — | 0.15 | — | 0.06 | — | — | 0.58 |
| Example 5 | 0 | d | — | — | — | — | 0.1 | — | — | — | 0.1 | — | 0.01 | — | — | 0.21 |
| | 10 | d | — | — | — | — | 0.23 | 0.03 | — | — | 0.16 | — | 0.05 | — | — | 0.47 |
| Example 6 | 0 | d | — | — | — | — | 0.08 | — | — | — | 0.08 | — | 0.01 | — | — | 0.17 |
| | 10 | d | — | — | — | — | 0.25 | 0.05 | — | — | 0.15 | — | 0.05 | — | 0.03 | 0.53 |
| Example 7 | 0 | d | — | — | — | — | 0.01 | — | — | — | — | — | — | — | — | 0.01 |
| | 10 | d | — | — | — | — | 0.19 | 0.03 | — | — | 0.09 | — | — | — | — | 0.31 |
| Control | 0 | d | 0.15 | 0.12 | — | 0.08 | 0.38 | — | — | — | — | — | 0.05 | 0.08 | 0.05 | 0.91 |
| group 1 | 10 | d | 0.26 | 0.32 | — | 0.15 | 0.53 | — | 0.05 | 0.08 | 0.21 | — | 0.1 | 0.12 | 0.12 | 1.94 |
| Control | 0 | d | 0.08 | — | 0.05 | 0.06 | 0.23 | — | 0.05 | 0.12 | 0.1 | — | 0.08 | 0.08 | 0.05 | 0.9 |
| group 2 | 10 | d | 0.38 | 0.45 | 0.05 | 0.24 | 0.85 | — | 0.06 | 0.32 | 0.26 | — | 0.18 | 0.21 | 0.15 | 3.15 |
| Control | 0 | d | 0.05 | 0.15 | 0.01 | — | 0.18 | — | 0.01 | 0.06 | 0.02 | — | 0.06 | 0.08 | 0.1 | 0.72 |
| group 3 | 10 | d | 0.28 | 0.35 | 0.15 | 0.23 | 1.05 | — | 0.09 | 0.15 | 0.25 | — | 0.15 | 0.21 | 0.25 | 3.16 |
| Control | 0 | d | 0.02 | 0.11 | 0.03 | 0.02 | 0.13 | — | — | 0.03 | — | 0.01 | 0.02 | 0.05 | 0.07 | 0.49 |
| group 4 | 10 | d | 0.21 | 0.23 | 0.35 | 0.15 | 0.89 | — | 0.1 | 0.21 | 0.26 | 0.03 | 0.21 | 0.12 | 0.12 | 2.84 |

TABLE 3
The results of the HPLC analysis for related substances in the samples stored at 40° C. for 3 months

| | | | RRT | | | | | | | | | | | | | |
|---|---|---|---|---|---|---|---|---|---|---|---|---|---|---|---|---|
| related substances | | | | | | | 0.83 | | | | | | | | | Total |
| (%) | | | 0.34 | 0.7 | 0.78 | 0.81 | (Impurity A) | 0.97 | 1.07 | 1.12 | 1.24 | 1.26 | 1.35 | 1.41 | 1.42 | impurities |
| Example 1 | 0 | d | — | — | — | — | 0.03 | — | — | — | 0.02 | — | — | — | — | 0.05 |
| | 3 | m | — | — | — | — | 0.15 | — | — | — | 0.03 | — | — | — | — | 0.18 |
| Example 2 | 0 | d | — | — | — | — | 0.05 | — | — | — | 0.02 | — | — | — | — | 0.07 |
| | 3 | m | — | — | — | — | 0.12 | — | — | — | 0.02 | — | — | — | — | 0.15 |
| Example 3 | 0 | d | — | — | — | — | 0.03 | — | — | — | 0.01 | — | 0.01 | — | — | 0.05 |
| | 3 | m | — | — | — | — | 0.18 | 0.02 | — | — | 0.05 | — | 0.06 | — | — | 0.31 |
| Example 4 | 0 | d | — | — | — | — | 0.1 | — | — | — | 0.1 | — | 0.01 | — | — | 0.21 |
| | 3 | m | — | — | — | — | 0.23 | 0.05 | — | — | 0.2 | — | 0.05 | — | — | 0.53 |
| Example 5 | 0 | d | — | — | — | — | 0.1 | — | — | — | 0.1 | — | 0.01 | — | — | 0.21 |
| | 3 | m | — | — | — | — | 0.15 | — | — | — | 0.1 | — | 0.05 | — | — | 0.3 |
| Example 6 | 0 | d | — | — | — | — | 0.08 | — | — | — | 0.08 | — | 0.01 | — | — | 0.17 |
| | 3 | m | — | — | — | — | 0.18 | — | — | — | 0.08 | — | 0.05 | — | 0.03 | 0.34 |
| Example 7 | 0 | d | — | — | — | — | 0.01 | — | — | — | — | — | — | — | — | 0.01 |
| | 3 | m | — | — | — | — | 0.1 | — | — | — | — | — | — | — | — | 0.1 |
| Control | 0 | d | 0.15 | 0.12 | — | 0.08 | 0.38 | — | — | — | — | — | 0.05 | 0.08 | 0.05 | 0.91 |
| group 1 | 3 | m | 0.31 | 0.35 | — | 0.09 | 0.58 | — | 0.06 | 0.08 | 0.2 | — | 0.11 | 0.1 | 0.15 | 2.03 |
| Control | 0 | d | 0.08 | — | 0.05 | 0.06 | 0.23 | — | 0.05 | 0.12 | 0.1 | — | 0.08 | 0.08 | 0.05 | 0.9 |
| group 2 | 3 | m | 0.45 | 0.36 | 0.08 | 0.2 | 0.95 | — | 0.16 | 0.35 | 0.28 | — | 0.2 | 0.25 | 0.32 | 3.6 |
| Control | 0 | d | 0.05 | 0.15 | 0.01 | — | 0.18 | — | 0.01 | 0.06 | 0.02 | — | 0.06 | 0.08 | 0.1 | 0.72 |
| group 3 | 3 | m | 0.36 | 0.38 | 0.25 | 0.29 | 1.07 | — | 0.12 | 0.16 | 0.2 | — | 0.18 | 0.2 | 0.22 | 3.43 |
| Control | 0 | d | 0.02 | 0.11 | 0.03 | 0.02 | 0.13 | — | — | 0.03 | — | 0.01 | 0.02 | 0.05 | 0.07 | 0.49 |
| group 4 | 3 | m | 0.42 | 0.2 | 0.39 | 0.25 | 1.02 | — | 0.1 | 0.25 | 0.15 | 0.06 | 0.15 | 0.18 | 0.15 | 3.32 |

2. Redispersion effect, particle size distribution, sedimentation volume ratio:
Redispersion effect: Adding an appropriate amount of water to the medicine bottle, shaking it slightly for 1 minute, and observing whether the particles are completely dispersed.
Particle size distribution: Taking an appropriate amount of this product, using a laser diffraction particle size analyzer, according to the particle size and particle size distribution measurement method (Ch.P 2015 Edition Volume IV General Chapters 0982 Third Method Wet Method), and using the saturated solution of amlodipine besylate as the dispersion medium to determine.
Sedimentation volume ratio: Taking an appropriate amount of this product, adding water to make a suspension containing about 1 mg of amlodipine per 1 ml, taking 50 ml of the solution, placing it in a graduated cylinder with a stopper, closing the stopper tightly, shaking vigorously for 1 minute, and letting stand for 45 minutes, The result should meet the requirements (Ch.P 2015 Edition Volume IV General Chapters 0123).
The specific results are shown in Table 4.

TABLE 4

| | | Particle size | | | Sedimen- |
|---|---|---|---|---|---|
| | | distribution | | | tation |
| Test | | D10 | D50 | D90 | volume |
| items | Redispersion effect | (μm) | (μm) | (μm) | ratio |
| Example | A small amount of | 7.5 | 25.6 | 53.2 | 0.99 |
| 1 | small particles did not | | | | |
| | dissolve immediately, | | | | |
| | no agglomeration, and | | | | |
| | many bubbles. after 10 | | | | |
| | minutes of storage, the | | | | |
| | particles dissolved. | | | | |
| Example | Easy to disperse, | 6.9 | 26.8 | 56.2 | 0.92 |
| 2 | basically free of small | | | | |
| | particles, no | | | | |
| | agglomeration, and a small | | | | |
| | amount of bubbles. | | | | |
| Example | Easy to disperse, | 7.8 | 27.5 | 55.3 | 0.90 |
| 3 | basically free of small | | | | |
| | particles, no | | | | |
| | agglomeration, and a small | | | | |
| | amount of bubbles. | | | | |
| Example | Easy to disperse, | 6.9 | 26.9 | 54.3 | 0.99 |
| 4 | basically free of small | | | | |
| | particles, no | | | | |
| | agglomeration, and a small | | | | |
| | amount of bubbles. | | | | |
| Example | Easy to disperse, no | 5.8 | 19.5 | 46.5 | 0.90 |
| 5 | small particles, no | | | | |
| | agglomeration, | | | | |
| | many bubbles. | | | | |
| Example | Easy to disperse, no | 10.6 | 35.2 | 60.3 | 0.99 |
| 6 | small particles, no | | | | |
| | agglomeration and a | | | | |
| | small amount of bubbles. | | | | |
| Example | Easy to disperse, no | 5.6 | 20.3 | 45.6 | 0.99 |
| 7 | small particles, no | | | | |
| | agglomeration, and a small | | | | |
| | amount of bubbles. | | | | |
| Control | / | 3.6 | 15.1 | 42.3 | 0.99 |
| group 1 | | | | | |
| Control | Easy to disperse, with a | 23.6 | 104.4 | 209.6 | 0.68 |
| group 5 | small amount of | | | | |
| | particle precipitation, no | | | | |
| | agglomeration, and a | | | | |
| | small amount of bubbles. | | | | |
| Control | Easy to disperse, no | 18.5 | 50.6 | 109.5 | 0.86 |
| group 6 | small particles, no | | | | |
| | agglomeration, and a small | | | | |
| | amount of bubbles. | | | | |

The results showed that the redispersion effect, particle size distribution, and sedimentation volume ratio of the samples prepared in the examples met the requirements. The amlodipine dry suspensions prepared by the stirring process and the ultrasonic stirring process had large particle size, wide particle size distribution, faster sedimentation, and the sedimentation volume ratio did not meet the requirements; the amlodipine dry suspensions prepared by the high-speed homogenization process had a smaller particle size, narrow particle size distribution, slower sedimentation, and the sedimentation volume ratio met the requirements.

1. A process for preparing amlodipine dry suspension, the process comprising: a) preparing an aqueous mixture 1 containing amlodipine besylate by using a high-speed homogenization method; b) adding sodium benzoate to the mixture 1, and homogenizing same at a high speed to obtain a mixture 2; and c) granulating the mixture 2 and other components, and then drying same; no surfactant is added during the process.

2. The process of claim 1, wherein the surfactant is polysorbate 80 or sodium lauryl sulfate.

3. The process of claim 1, wherein the rotating speed of the high-speed homogenization is between 4000 rev/min and 10000 rev/min, and the homogenization is performed for between 2 minutes and 4 minutes.

4. The process of claim 1, wherein the weight percentage of the amlodipine besylate in the aqueous mixture 1 in the step a) is between 1% and 5%.

5. The process of claim 1, wherein the step c) granulation method is high shear wet granulation or fluidized bed granulation.

6. The process of claim 1, wherein the weight percentage of the amlodipine besylate in the dry suspension is between 0.5% to 2%, the ratio of the weight of sodium benzoate to amlodipine besylate is between 1:1 and 5:1.

7. An amlodipine dry suspension obtained by the process of claim 1, wherein comprises amlodipine besylate, sodium benzoate, a diluent, and a suspending agent.

8. The dry suspension of claim 7, wherein the diluent comprises mannitol, erythritol or maltitol, or combinations thereof, the weight percentage of the diluent in the dry suspension is between 80% and 95%.

9. The dry suspension of claim 7, wherein the suspending agent is hypromellose K4M, and the weight percentage of the suspending agent in the dry suspension is between 1% and 5%.

10. The dry suspension of claim 7, wherein further comprises a pharmaceutically acceptable defoamer, a binder, a glidant and a flavoring agent.

11. The process of claim 4, wherein the weight percentage of the amlodipine besylate in the dry suspension is between 0.5% to 2%, the ratio of the weight of sodium benzoate to amlodipine besylate is between 1:1 and 5:1.
